(19) United States
(12) Patent Application Publication
(10) Pub. No.: US 2017/0307579 A1
(43) Pub. Date: Oct. 26, 2017

(54) AMINE COMPOUND SENSING MARKER

(71) Applicant: TOSHIBA TEC KABUSHIKI KAISHA, Tokyo (JP)

(72) Inventor: Ryozo Akiyama, Mishima Shizuoka (JP)

(21) Appl. No.: 15/492,207

(22) Filed: Apr. 20, 2017

(30) Foreign Application Priority Data
Apr. 22, 2016 (JP) 2016-086392

(51) Int. Cl. G01N 33/12

(52) U.S. Cl. CPC G01N 33/12

(57) ABSTRACT

Provided herein is an amine compound sensing marker capable of easily sensing an amine compound with high sensitivity. An amine compound sensing marker of an exemplary embodiment includes a sensing body as a retention medium where a composition containing an aggregative phosphor and a solvent is provided, the aggregative phosphor being a phosphor that aggregates and shows a fluorescence characteristics change in the presence of an amine compound. The retention medium is processed from a glass fiber.

### CROSS-REFERENCE TO RELATED APPLICATION

This application is based upon and claims the benefit of priority from Japanese Patent Application No. P2016-086392, filed Apr. 22, 2016, the entire contents of which are incorporated herein by reference.

### FIELD

Embodiments described herein relate generally to a sensing marker capable of easily sensing an amine compound with high sensitivity.

### BACKGROUND

In the natural environment we live in, there is a wide range of compounds, both artificial and natural, that pose health hazard to the human body. Such artificial hazardous compounds are produced in manufacturing processes of industrial products, or contained in the products themselves. The naturally occurring compounds include those that generate from animals and plants themselves, and those that occur in the proliferation of microorganisms such as bacteria and fungi. Evidently, measures are taken to impose quantitative limits on these compounds.

Amine compounds are an example of the compounds that pose health hazard to the human body. For example, N-nitrosoamines in rubber products occur as the secondary amines that generate through decomposition of the vulcanization accelerator added in rubber production partly reacts with the nitrite or other nitrogen oxides occurring in the environment or in the body, or with nitrogen oxides used for rubber production. Some N-nitrosoamines are carcinogens. In Europe, regulations are imposed on the level of N-nitrosoamine release from rubber teats and dummies.

Melamine in resin products is a material used to produce melamine resin, and EU regulates the level of melamine release from resin products. Triethylamine and tributylamine are catalysts used for polycarbonate production. The Food Sanitation Act in Japan regulates amine contents in polycarbonate products. Other examples of hazardous amine compounds include carcinogenic aromatic amines that are produced by decomposition of inorganic nitrogens NH₃—N (ammonia nitrogen), NO₂—N (nitrite nitrogen), and NO₃—N (nitrate nitrogen) involved in water contamination, or by decomposition of animal tissue components such as organic nitrogen, proteins, amino acids, and polypeptides, and urea nitrogen contained in the decomposition process of these components, and pigment components such as a dye.

Amine compounds are potential analytes in a wide range of fields. Particularly, amine compounds that generate in food products have potential use as an index of food freshness, and a simple method for detection of amine compounds is needed.

A method using a tetraphenylethene phosphor is known that enables easy and quick detection of an amine compound in a food product. In this method, the tetraphenylethene phosphor is contacted with an amine compound in a solution, and an increased fluorescence intensity by interaction of these compounds is detected for the detection of an amine compound.

### DESCRIPTION OF THE DRAWINGS

FIGS. 1A and 1B are diagrams representing an example of a sensing marker, and an example of use of the sensing marker according to First Embodiment.

FIGS. 2A to 2C are diagrams representing an example of an amine compound sensing process by a sensing marker according to Second Embodiment.

FIG. 3 represents the relationship between the hydrophobicity parameter Log P values of a glycol-based solvent and a biogenic amine.

FIGS. 4A to 4C are diagrams explaining the fabrication method of the evaluation sample according to Example.

FIGS. 5A to 5C are diagrams explaining the fabrication method of the evaluation sample according to Comparative Example.

FIG. 6 is a diagram explaining an evaluation method of an evaluation sample.

FIG. 7 shows images of the fluorescence state of the evaluation sample according to Comparative Example observed overtime for samples using a food product and for samples using purified water.

FIG. 8 shows images of the fluorescence state of the evaluation sample according to Example observed over time for samples using a food product and for samples using purified water.

FIG. 9 shows images of the fluorescence state of the evaluation sample according to Example observed over time for samples using a food product.

FIG. 10 shows images of the fluorescence state of the evaluation sample according to Example observed over time for samples using purified water.

### DETAILED DESCRIPTION

While the foregoing method enables easy and quick detection of an amine compound, the fluorescence intensity may be affected by non-amine components, for example, moisture, and, depending on the environment, the detection sensitivity for amine compounds becomes weak.

The present invention was made to provide a solution to the foregoing problem, and it is an object of the present invention to provide an amine compound sensing marker capable of easily sensing an amine compound with high sensitivity, without influence of moisture.

An amine compound sensing marker according to an exemplary embodiment includes a sensing body as a retention medium where a composition containing an aggregative phosphor and a solvent is provided, the aggregative phosphor being a phosphor that aggregates and shows a fluorescence characteristics change in the presence of an amine compound. The retention medium is processed from a glass fiber.

Embodiments are described below in detail with reference to the accompanying drawings.

### First Embodiment

A basic form of an amine compound sensing marker (hereinafter, also referred to simply as “sensing marker”) according to an embodiment is a label that includes: an aggregative phosphor that aggregates and shows a fluorescence characteristics change in the presence of an amine compound; a solvent dissolving the aggregative phosphor; and a sensing body configured from a retention medium retaining the aggregative phosphor and the solvent, the retention medium being processed from a glass fiber. As used herein, “sensing body” refers to a region where an aggregative phosphor solution is supported (retained) by the retention medium.

The amine compound sensing marker according to the embodiment does not show a fluorescence characteristics change due to moisture, and enables sensing an amine compound with high sensitivity even in a high-humidity environment. The sensing marker is therefore useful as, for example, a marker that is installed inside a sealed container with a food product, and that checks (or determines) the freshness or the spoilage of the food product by sensing amines (hereinafter, also referred to as “biogenic amines”) generated by food spoilage.

### Biogenic Amine

If left unchecked, food typically undergoes changes in quality such as smell, appearance, texture, and taste overtime before it is no longer suited for consumption. Such changes in food quality are called deterioration, decaying, or degradation, or, more commonly, “food spoilage.” Food deterioration is caused by microorganisms, insects, or self digestion. It also has chemical (lipid oxidation, browning), and physical (damage such as cuts and crushes) causes. In many cases, food deterioration is caused by proliferation of microorganisms (putrefactive bacteria). Such deterioration of food by proliferation of microorganisms often makes food inedible, and this is broadly defined as “spoilage.”

Spoilage refers to the process by which protein in food decomposes by the effects of microorganisms, and produces harmful substances or a bad odor. This is often distinguished from “decaying” or “degradation”, which describes a state in which carbohydrates and fats decompose by the effects of microorganisms, and produce a bad flavor not suitable for consumption. The main components of a foul odor are ammonia, and various amine components such as trimethylamine, or biogenic amines as they are also called.

It is accordingly useful to quantify the biogenic amine component to find the extent of spoilage in food products rich in protein, such as in meat and fish. Detection by, for example, high-performance liquid chromatography is commonly used as a quantitative biogenic amine analysis method. However, the method requires a complicated pretreatment, and a long measurement time for a sample, and takes time to determine the result, in addition to being costly.

The nitrogen compounds in food are mainly proteins, which become hydrolyzed by the enzymes of microorganisms and food into polypeptides, and to simple peptides or amino acids. These amino acids decompose through reactions such as deamination, transamination, and decarboxylation, and produce biogenic amines.

Examples of the biogenic amines produced by amino acids include 1,2-ethylenediamine, 1,3-propanediamine, 1,4-butanediamine, 1,5-pentanediamine, 1,6-hexanediamine, spermidine, spermine, histamine, and tryptamine.

### Aggregative Phosphor

The aggregative phosphor according to the embodiment refers to a phosphor that aggregates, or precipitates into crystals in the presence of an amine compound, and changes its fluorescence characteristics, including the profile and the intensity of the fluorescence or excitation spectrum, and fluorescence lifetime. The aggregation-induced emission molecule described in JP-A-2012-51816 is an example of such an aggregative phosphor. The aggregation-induced emission molecule does not fluoresce in a dissolved state in a solvent even when exposed to excitation light , but emits fluorescence upon forming aggregates under excitation light. A specific example is the tetraarylethene derivative represented by the following general formula (I).

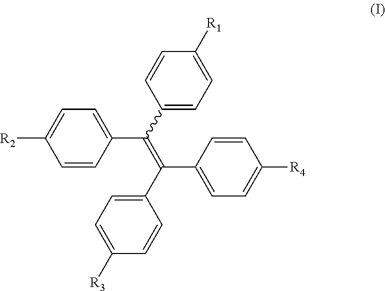

wherein R₁, R₂, R₃, and R₄ are each independently selected from —COOM₁, —(CH₂)ₘ—COOM₂, —X—(CH₂)ₙ—COOM₃, —Y—(CH₂)ₒ—Z—(CH₂)ₚ—COOM₄ (where M₁, M₂, M₃, and M₄ each independently represent a hydrogen atom or a cation, X, Y, and Z each independently represent —O—, —NH—, or —S—, and m, n, o, and p each independently represent an integer of 1 to 6), a hydrogen atom, a halogen atom, a hydroxyl group, a nitro group, a carbamoyl group, an alkyl group of 1 to 6 carbon atoms, a haloalkyl group of 1 to 6 carbon atoms, an alkenyl group of 2 to 6 carbon atoms, a cycloalkyl group of 3 to 10 carbon atoms, an alkyloxy group of 1 to 6 carbon atoms, an acyl group of 2 to 6 carbon atoms, an amino group, an alkylamino group of 1 to 6 carbon atoms, an aryl group of 6 to 10 carbon atoms, and an a heteroaryl group of 5 to 10 carbon atoms, and at least two of R₁, R₂, R₃, and R₄ are each independently selected from —COOM₁, —(CH₂)ₘ—COOM₂, —X—(CH₂)ₙ—COOM₃, and —Y—(CH₂)ₒ—Z—(CH₂)ₚ—COOM₄ (here, M₁, M₂, M₃, M₄, X, Y, Z, m, n, o, and p are as defined above).

In the formula, “cation” is not particularly limited, and may be an organic cation or an inorganic cation. Examples of such cations include ammonium, alkali metals, alkali earth metals, and pyridinium. The cations maybe different when two or more cations are present within the molecule.

The tetraarylethene derivative represented by the general formula (I) aggregates as it becomes less soluble in solution through hydrogen bonding or electrostatic interaction (hereinafter, also referred to as “reaction”) of the carboxyl group in the molecule with the amine compound. The aggregated tetraarylethene derivative emits fluorescence when exposed to excitation light such as ultraviolet (UV) light. In the embodiment, the composition (mixture) containing the aggregative phosphor and the solvent retained in the retention medium is prepared in such a concentration that the unreacted aggregative phosphor does not aggregate or precipitate, or does not become saturated.

### Solvent

The solvent according to the embodiment is a solvent capable of dissolving the aggregative phosphor, and that does not undergo evaporative volume reductions in the atmosphere over a certain length of time. Further, because the sensing marker is used by being attached to food or being installed in the vicinity of food in food applications, the solvent is selected preferably from solvents that are safe to the human body. The solvent according to the embodiment may be a mixed solvent of two or more solvents.

The solvent is, for example, a glycol-based solvent having a high boiling point and low toxicity. Specific examples include ethylene glycol-based solvents such as polyethylene glycol monomethyl ether, diethylene glycol ethyl methyl ether, polyethylene glycol dimethyl ether, triethylene glycol butyl methyl ether, and diethylene glycol butyl methyl ether; and propylene glycol-based solvents such as propylene glycol monomethyl ether, propylene glycol monobutyl ether, and propylene glycol dimethyl ether.

### Retention Medium

The retention medium according to the embodiment retains a composition (mixture) containing the aggregative phosphor and the solvent. Considering retention of the composition (mixture), the retention medium is one with a certain level of porosity. In the embodiment, the retention medium is a filter medium processed from a glass fiber. By using a filter medium processed from a glass fiber, changes in fluorescence characteristics due to moisture can be prevented.

The filter medium processed from a glass fiber may be any of a variety of filter media that differ in, for example, the glass fiber diameter, the type of the hydrophilic or hydrophobic treatment used, or the presence or absence of a binder. Preferred is a glass-fiber filter paper containing an acrylic resin as an organic binder.

Desirably, the composition (mixture) is impregnated in the retention medium in about the same amount as the pore volume of the retention medium. The reaction rate between the amine compound and the aggregative phosphor increases as the amount of amine compound increases, and the aggregative phosphor forms a larger amount of aggregates. This brings about fluorescence characteristics changes, for example, an increased fluorescence intensity. Specifically, there is a correlation between the amount of generated amine compound, and the fluorescence characteristics. Accordingly, the fluorescence quantity, which has a correlation with the amount of the amine that reacts with the aggregative phosphor, becomes more accurate by specifying the amount of the composition (mixture) impregnated in the pores.

### Base Material

The amine compound sensing marker according to the embodiment may use a base material that supports the retention medium, as required. Preferably, the base material is selected from materials that are resistant to the solvent dissolving the aggregative phosphor, and that do not emit fluorescence by themselves. However, the base material is not particularly limited, and any material may be used with the provision that the fluorescence wavelength is not close to the fluorescence wavelength of the aggregative phosphor emitting fluorescence.

Examples of such base materials include plastic sheets such as a Teflon® sheet, a polyimide sheet, a polyester film, a polyacetal sheet, a nylon sheet, a polycarbonate sheet, a polypropylene sheet, a polyethylene sheet, a PET film, and a vinyl chloride sheet; and glass plates.

### Determination Method

In the embodiment, the amine compound sensing marker according to First Embodiment is kept with a sample such as a food product over a certain time period, and the fluorescence emitted by the sensing marker upon UV irradiation by a ultraviolet source unit is confirmed with an emission detector to determine the state of the sample, for example, the freshness of the food product. Here, the emission detector means visual inspection by naked eye, or an imaging device such as a digital camera.

In the embodiment, it is desirable to make an observation in the dark by avoiding visible light when determination with the emission detector is made by visual inspection with naked eye. The accuracy of determination can improve when a fluorescence photometer is used. The accuracy of determination can further improve when observation is made by checking the image (image pattern) produced by a CCD or a CMOS image sensor of devices such as a digital camera.

The electronic image produced by a digital camera or other such devices may be processed so that a weak fluorescence image is converted into a higher contrast image. Such image processing is effective when a slight difference in fluorescence intensity, specifically, a slight difference in the amount of amine compound needs to be distinguished. It is also possible to use, for example, a smartphone with a camera having a colorimetric function. In this way, the freshness can be determined by automatically distinguishing the difference.

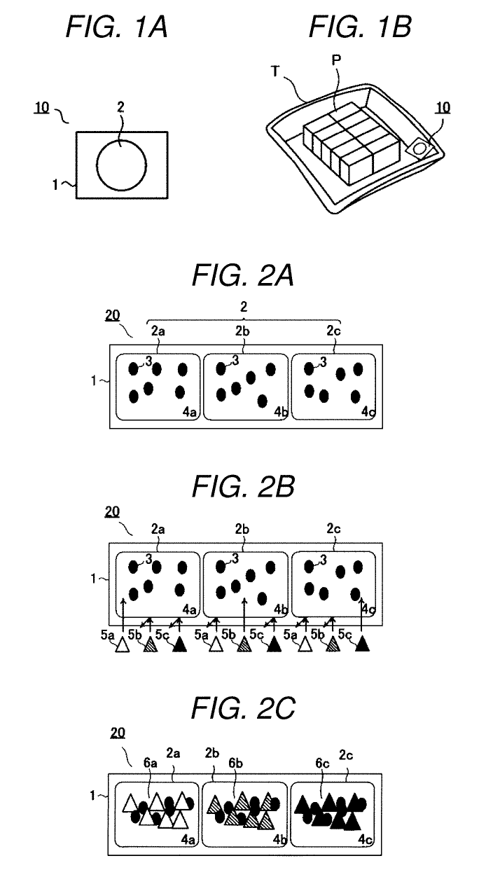

An example of use of the sensing marker according to the embodiment is described below. FIG. 1A shows an example of the sensing marker according to First Embodiment. FIG. 1B shows an example of use of the sensing marker.

As shown in FIG. 1A, a sensing marker 10 according to First Embodiment includes a sheet-like base material 1, and a filter medium 2 (retention medium), processed from a glass fiber, supported on the base material 1. The filter medium 2 is impregnated with a mixture of the aggregative phosphor dissolved in a solvent. In other words, the sensing marker 10 according to First Embodiment includes a retention medium layer retaining the aggregative phosphor mixture, and a base material layer supporting the retention medium layer.

As shown in FIG. 1B, the sensing marker 10 according to First Embodiment is placed on a food tray T with a food product P, and sealed with, for example, a wrapping film wrapping the food tray T. As the food product P spoils, the food produces a biogenic amine. The biogenic amine diffused in the food tray T reacts with the aggregative phosphor retained in the retention medium 2 of the sensing marker 10, and the aggregative phosphor starts to aggregate. In response to UV light applied to the sensing marker 10, the aggregates of the aggregative phosphor emit fluorescence, allowing a user to recognize the spoiled state of the food product.

### Second Embodiment

A sensing marker according to Second Embodiment includes an aggregative phosphor that aggregates and shows a fluorescence characteristics change in the presence of an amine compound, a solvent dissolving the aggregative phosphor, and a sensing body configured from a retention medium processed from a glass fiber and retaining the aggregative phosphor and the solvent. The sensing body is demarcated into at least two parts, and solvents with different hydrophobic properties are retained in the demarcated sensing bodies.

The amine compounds to be sensed have different hydrophobic properties. Accordingly, the sensing marker according to First Embodiment can only sense amine compounds that are compatible with the solvent used, and cannot sense amine compounds of greatly different hydrophobic properties because such amine compounds cannot dissolve in the solvent. That is, the sensing marker according to First Embodiment is limited in the type of amine compound it can sense. In contrast, the sensing marker according to Second Embodiment retains solvents of different hydrophobic properties in different sensing bodies, and can sense many more different amine compounds.

FIGS. 2A to 2C are diagrams representing an example of an amine compound sensing process by the sensing marker according to Second Embodiment. As shown in FIG. 2A, a sensing marker 20 according to Second Embodiment includes retention media 2a, 2b, and 2c that are supported as demarcated sensing bodies on a base material 1. The retention media 2a, 2b, and 2c are retaining the aggregative phosphor 3, and solvents 4a, 4b, and 4c of different hydrophobic properties, respectively.

As shown in FIGS. 2B and 2C, for example, amine compounds 5a, 5b, and 5c generated from a spoiled food product react with the aggregative phosphor 3 in the sensing marker 20 according to Second Embodiment, and form aggregates 6a, 6b, and 6c in the retention media 2a, 2b, and 2c retaining the solvents 4a, 4b, and 4c that more easily dissolve the amine compounds 5a, 5b, and 5c, respectively.

The solvents used for the sensing marker according to Second Embodiment are appropriately selected taking into account the hydrophobicity for the amine compound. Hydrophobicity parameter Log P value is an example of an index of hydrophobicity. Here, the hydrophobicity parameter Log P value represents the partition coefficient of a substance in water and 1-octanol.

Amine compounds with hydrophobicity parameter Log P values closer to the hydrophobicity parameter Log P value of the solvent are more likely to dissolve in the solvent, and an amine compound becomes less likely to dissolve in the solvent as the hydrophobicity parameter Log P value of the amine compound becomes more different from the hydrophobicity parameter Log P value of the solvent. That is, substances with similar hydrophobicity parameter Log P values are more compatible with each other. Having similar hydrophobicity parameter Log P values for the solvent and the amine compound means that the solvent and the amine compound are more likely to form a homogenous mixture.

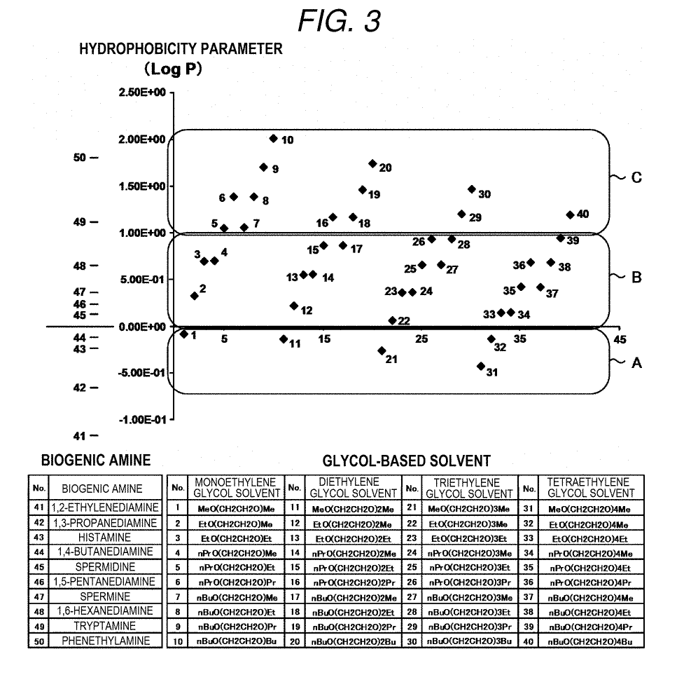

FIG. 3 represents an example of the correlation of hydrophobicity parameter Log P values between amine compounds and glycol-based solvents, and how the hydrophobicity parameter Log P values of the solvents compare to the hydrophobicity parameter Log P values of the amine compound biogenic amines. The hydrophobicity parameter Log P values were calculated using the Molecular Modeling Pro. Plus software, version 7.0.4, available from Norgwyn Montgomery Software.

As shown in FIG. 3, the hydrophobicity parameter Log P values of the glycol-based solvents are highly dependent on the terminal substituent, and the number of repeating glycol units. Specifically, the hydrophobicity parameter Log P value increases as the total number of carbon atoms in the terminal substituent increases, and decreases as the number of repeating glycol units increases.

In the case of the glycol-based solvents shown in FIG. 3, it is preferable from the relationship with the hydrophobicity parameter Log P values of the biogenic amines to dispose, for example, the following sensing bodies in the following order.

(1) A sensing body in which the solvent is a monoethylene glycol or diethylene glycol solvent with a terminal substituent containing 2 carbon atoms in total, or in which the solvent is a triethylene glycol solvent with a terminal substituent containing 2 to 3 carbons in total, or a tetraethylene glycol solvent with a terminal substituent containing 2 to 5 carbon atoms in total (A in FIG. 3),

(2) A sensing body in which the solvent is a monoethylene glycol solvent with a terminal substituent containing 3 to 4 carbon atoms in total, a diethylene glycol solvent with a terminal substituent containing 3 to 6 carbon atoms in total, a triethylene glycol solvent with a terminal substituent containing 3 to 6 carbon atoms in total, or a tetraethylene glycol solvent with a terminal substituent containing 3 to 7 carbon atoms in total (B in FIG. 3),

(3) A sensing body in which the solvent is a monoethylene glycol solvent with a terminal substituent containing 5 to 8 carbon atoms in total, a diethylene glycol solvent with a terminal substituent containing 6 to 8 carbon atoms in total, a triethylene glycol solvent with a terminal substituent containing 7 to 8 carbon atoms in total, or a tetraethylene glycol solvent with a terminal substituent containing 7 to 8 carbon atoms in total (C in FIG. 3).

This makes the sensing body A more sensitive to 1,3-propanediamine (no. 42), histamine (no. 43), and 1,4-butanediamine (no. 44), the sensing body B more sensitive to spermidine (no. 45), 1,5-pentanediamine (no. 46), spermine (no. 47), and 1,6-hexanediamine (no. 48), and the sensing body C more sensitive to triptophan (no. 49), and phenethylamine (no. 50). The sensing marker according to Second Embodiment can sense a wider range of amine compounds.

### EXAMPLES

The exemplary embodiments are described below in greater detail using Examples and Comparative Example. The following descriptions are not to be construed as limiting.

### Example 1, and Comparative Example

Preparation of Fluorescence Solution (Composition)

The compound (1) of the general formula (I) of when R₁ and R₃ are carboxyl groups, and R₂ and R₄ are hydrogen atoms was used as the aggregative phosphor, and the aggregative phosphor was dissolved in the solvent below in a concentration of 0.02 weight % to prepare a fluorescence solution A.

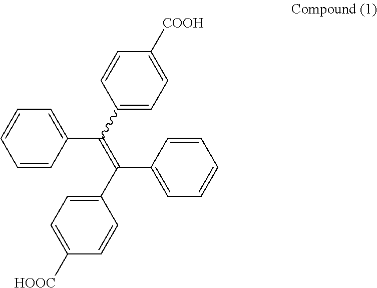

### Fluorescence Solution A

Polyethylene glycol dimethyl ether: 99.98 wt % (Hisolv MPM, Toho Chemical Industry Co., Ltd.)

Compound (1): 0.02 wt %

### Fabrication of Evaluation Sample

### Evaluation Sample 100 According to Example 1

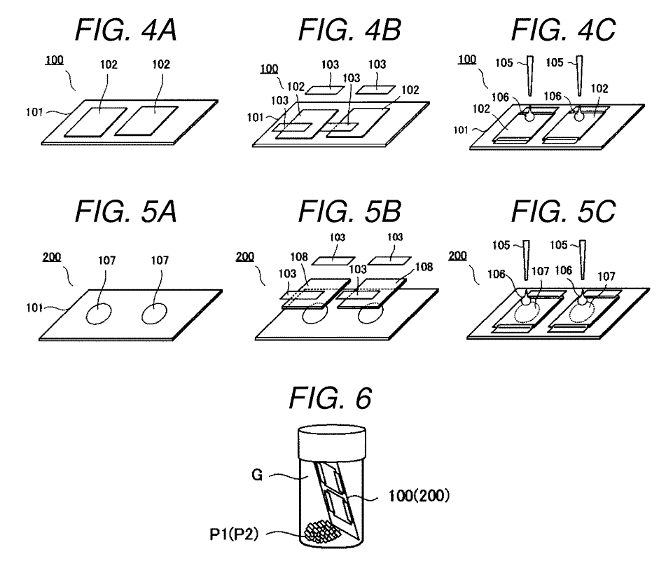

FIGS. 4A to 4C are diagrams explaining the fabrication method of the evaluation sample according to Example 1.

First, as shown in FIG. 4A, two glass filters 102 were installed on a glass sheet 101, and the both ends of the glass filters 102 were fixed with a polyimide adhesive tape 103, as shown in FIG. 4B. A glass filter paper containing an organic binder (acrylic resin; Advantec product GS-25, thickness 0.21 mm) was used as the glass filter 102.

Thereafter, as shown in FIG. 4C, a fluorescence solution 106 (about 10 μL of the fluorescence solution A) was dropped onto the glass filters 102 with a pipette 105, and the glass filters 102 were impregnated with the fluorescence solution 106 to provide sensing bodies, and obtain an evaluation sample 100 according to Example 1 (hereinafter, also referred to simply as “evaluation sample 100”).

### Evaluation Sample 200 According to Comparative Example

FIGS. 5A to 5C are diagrams explaining the fabrication method of the evaluation sample according to Comparative Example.

First, as shown in FIG. 5A, two membrane filters 107 (Cellulose Acetate CO20A available from Advantec; 0.125 ml) were installed on a glass sheet 101. Thereafter, as shown in FIG. 5B, a screen mesh 108 (available from Murakami Co., Ltd.; thread diameter: 34 μm; thickness: 52 μm) was placed over each membrane filter 107, and the both ends of the screen mesh 108 were fixed with the polyimide adhesive tape 103.

Thereafter, as shown in FIG. 5C, a fluorescence solution 106 (about 10 μL of the fluorescence solution A) was dropped onto the membrane filters 107 through the screen mesh 108, and the membrane filters 107 were impregnated with the fluorescence solution 106 to provide sensing bodies, and obtain an evaluation sample 200 according to Comparative Example (hereinafter, also referred to simply as “evaluation sample 200”).

### Sample Evaluation

As illustrated in FIG. 6, the evaluation sample 100 of Example 1 and the evaluation sample 200 of Comparative Example fabricated in the manner described above were separately installed in a lidded glass container G containing a sample (fresh food product P1, kamaboko, minced and steamed fish meat, or a waste P2 impregnated with 0.5 ml of purified water). Here, the evaluation sample was installed without contacting the sensing bodies to the sample. After installing the evaluation sample, the container was closed, and stored in a room temperature environment. The evaluation samples 100 and 200 were then observed for fluorescence state over a time period.

The fluorescence observation was conducted by observing a digital camera image of the fluorescence state after the evaluation samples 100 and 200 were taken out of the glass container G, and irradiated with UV light from the glass sheet 101 side under suitable dark-room conditions. UV light was applied by using a hand-held black light.

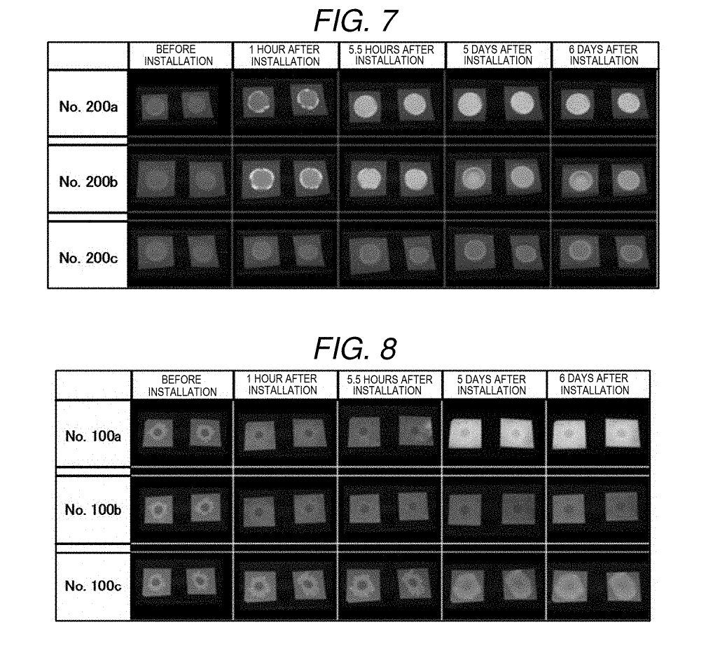

FIG. 7 shows images of the fluorescence state of the evaluation sample 200 according to Comparative Example before installation, and after about 1 hour and about 5.5 hours, and after 5 days and 6 days from installation.

Sample No. 200a is an image of the evaluation sample installed in a glass container G containing the fresh food product P1 (kamaboko; minced and steamed fish meat). Sample No. 200b is an image of the evaluation sample installed in a glass container G containing the waste P2 impregnated with 0.5 ml of purified water. Sample No. 200c is an image of the evaluation sample installed in an empty glass container G that did not contain the fresh food product P1 or the waste P2 impregnated with purified water.

By comparing the images of sample No. 200a to 200c, fluorescence can be observed after 1 hour in sample No. 200a installed in the glass container G containing the fresh food product P1. The same fluorescence observed in sample No. 200a installed in the glass container G containing the fresh food product P1 was also observed in sample No. 200b installed in the glass container G containing the waste P2 impregnated with 0.5 ml of purified water.

FIG. 8 shows images of the fluorescence state of the evaluation sample 100 according to Example 1 before installation, and after about 1 hour and about 5.5 hours, and after 5 days and 6 days from installation.

Sample No. 100a is an image of the evaluation sample installed in a glass container G containing the fresh food product P1 (kamaboko; minced and steamed fish meat). Sample No. 100b is an image of the evaluation sample installed in a glass container G containing the waste P2 impregnated with 0.5 ml of purified water. Sample No. 100c is an image of the evaluation sample installed in an empty glass container G that did not contain the fresh food product P1 or the waste P2 impregnated with 0.5 ml of purified water.

By comparing the images of sample No. 100a to 100c, strong fluorescence was observed after 5 days and after 6 days from the installation of sample No. 100a in the glass container G containing the fresh food product P1, whereas fluorescence was observed neither in sample No. 100b installed in the glass container G containing the waste P2 impregnated with 0.5 ml of purified water, nor in sample No. 100c installed in the empty glass container G.

As demonstrated above, the effect of moisture on fluorescence intensity change was absent in the evaluation sample 100 according to Example 1.

### Examples 2 to 4

### Fabrication of Evaluation Sample

Evaluation samples 101, 102, and 103 according to Examples 2 to 4 were fabricated in the same manner as for the evaluation sample 100 according to Example 1, expect that the following glass-fiber filter paper was used as the glass filter 102.

### Evaluation Sample 101 According to Example 2

Glass-fiber filter paper containing no organic binder (glass-fiber filter paper GS-50 available from Advantec; thickness 0.19 mm)

### Evaluation Sample 102 According to Example 3

Glass-fiber filter paper containing an organic binder (acrylic resin) (glass-fiber filter paper GC-90 available from Advantec; thickness 0.30 mm)

### Evaluation Sample 103 According to Example 4

Glass-fiber filter paper containing an organic binder (acrylic resin) (glass-fiber filter paper DP-70 available from Advantec; thickness 0.52 mm)

### Sample Evaluation

The evaluation samples 101, 102, and 103 according to Examples 2 to 4 were installed in a lidded glass container G containing a sample (fresh food product P1, kamaboko, minced and steamed fish meat, or a waste P2 impregnated with 0.5 ml of purified water). The evaluation sample 100 according to Example 1 was also installed for comparison. Here, the evaluation samples were installed without contacting the sensing bodies to the sample. After installing the evaluation sample, the container was closed, and stored in a room temperature environment. The evaluation samples 100, 101, 102, and 103 were then observed for fluorescence state over a time period. Variation between the samples can be ignored by installing and evaluating the evaluation samples in the same glass container G in this fashion.

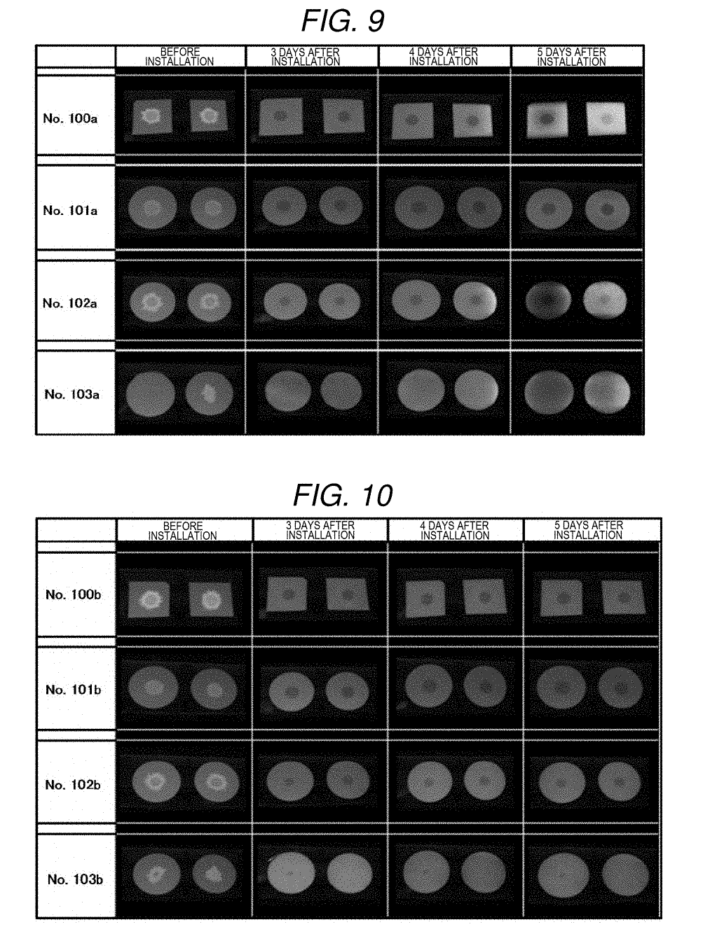

FIGS. 9 and 10 show images of the fluorescence state of the evaluation samples 100, 101, 102, and 103 before installation, and after 3 days, 4 days, and 5 days from installation. Sample No. 100a to 103a in FIG. 9 are images of the evaluation samples 100 to 103 installed in the glass container G containing the fresh food product P1 (kamaboko; minced and steamed fish meat). Sample No. 100b to 103b in FIG. 10 are images of the evaluation samples 100 to 103 installed in the glass container G containing the waste P2 impregnated with 0.5 ml of purified water.

By comparing the images of sample No. 100a to 103a installed in the glass container G containing the food product P1 (kamaboko; minced and steamed fish meat) with the images of sample No. 100b to 103b installed in the glass container G containing the waste P2 impregnated with 0.5 ml of purified water, fluorescence was detected in the images of sample No. 100a to 103a, but not in the images of sample No. 100b to 103b. As demonstrated above, the effect of moisture on fluorescence intensity change was absent also in the evaluation samples 101 to 103 according to Examples 2 to 4.

By comparing the evaluation samples 100 to 103 according to Examples 1 to 4, fluorescence was observed after 4 days from installation in sample No. 100a, 102a, and 103a according to Examples 1, 3, and 4 using the glass-fiber filter paper containing an organic binder, as shown in FIG. 9, whereas sample 101a according to Example 2 using the glass-fiber filter paper containing no organic binder started to glow after 5 days from installation. As can be seen from these results, the samples according to Examples 1, 3, and 4 have higher detection sensitivity for the spoilage component than the sample according to Example 2.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An amine compound sensing marker comprising a sensing body as a retention medium where a composition containing an aggregative phosphor and a solvent is provided, the aggregative phosphor being a phosphor that aggregates and shows a fluorescence characteristics change in the presence of an amine compound,
wherein the retention medium is processed from a glass fiber.

2. The sensing marker according to claim 1, wherein the retention medium contains an organic binder.

3. The sensing marker according to claim 1, wherein the retention medium is glass-fiber filter paper.

4. The sensing marker according to claim 1, wherein the amine compound is volatile.

5. The sensing marker according to claim 1, wherein the amine compound is an amine that generates as a result of food spoilage.
